(19) United States
(12) Patent Application Publication
(10) Pub. No.: US 2003/0078526 A1
(43) Pub. Date: Apr. 24, 2003

(54) Relating to the control of urinary continence

(76) Inventor: Michael Farley, Indialantic, FL (US)

Correspondence Address: BIDYUT K. NIYOGI, 1900 BUILDING, SUITE 301, 1900 S. HARBOR CITY BLVD., MELBOURNE, FL, 32901, US

(21) Appl. No.: 09/752,965

(22) Filed: Dec. 29, 2000

(51) Int. Cl. A61H 9/00; A61H 19/00; A61M 29/02

(52) U.S. Cl. 601/148; 606/193; 601/45; 128/836

(57) ABSTRACT

A device for regulating and controlling urinary incontinence, comprising a fluid pump, a rigid chamber interconnected to the pump having an outlet for fluid flow, and extension member connected to said outlet, the extension member is of a pliable material and cylindrical in shape and adapted for insertion into a woman's vagina, to enable a woman's pelvic muscles to be exercised and toned.

This invention relates to a method of or a device for the control of urinary incontinence, and specifically, to a device that either stops or help prevent incontinence, using fluid pressure adjustments to the appropriate pelvic floor muscles.

In the specifications of U.S. Pat. No. 4,241,912 to Mercer et al dated Dec. 30, 1980 it was disclosed a vaginal exercise device and corresponding method for its use so as to improve muscle tone of the muscles of the pelvic diaphragm, which is incorporated herein as a reference.

Furthermore, the specifications of U.S. Pat. Nos. 4,050,449; 5,354,250 and 4,685,474 all disclosing related devices and are incorporated herein by way of references.

Exercises for improving the muscles of a female patient's pelvic/diaphragm have long been recognized as being beneficial in many ways for women's health. The major difficulty with teaching such exercises has been isolating and recognizing the muscles to be contracted. Resistance against these muscles has been shown to be helpful in recognizing the appropriate pelvic floor muscles to exercise. Resistance exercise is also recognized as one of the fastest and most effective methods of rapidly developing muscle strength.

The present invention offers three distinct advantages over other devices. The present invention provides a centering groove that positions and places the muscles in the proper position with respect to the device. Using fluid pressure resistance against the pelvic floor muscles by pumping fluid into a chamber, or decreasing fluid pressure by regulating a valve, the device is adjustable in both diameter and shape, enabling it to form a custom fit with fluid pressure on a soft rubber sleeve, and gives resistance against the muscles during exercise, along with adjustment to fluid pressure.

The present invention includes a device for regulating and controlling urinary incontinence, comprising a fluid pumping means; a rigid chamber interconnected to the fluid pumping means, said chamber having an outlet for fluid; and

extension member connected to said outlet for fluid flow, characterized in that said extension member includes an outer wall of a flexible material and substantially cylindrical in shape and adapted for insertion into a woman's vagina, so that the cylindrical extension member when located within the woman's vagina enables pelvic muscles to be exercised and toned.

This invention also includes a method for controlling and regulating urinary incontinence characterized by the steps of insertion of an exercise device, comprising a fluid pumping means, a rigid chamber interconnected to the fluid pumping means have an outlet for fluid; an extension member connected to said outlet for fluid flow, characterized in that said extension member is of a flexible material and substantially cylindrical in shape and adapted for insertion into a woman's vagina, so that the cylindrical extension member when located within the woman's vagina enables pelvic muscles to be exercised and toned.

The invention will now be described by way of example, with reference to the accompanying drawings in which:

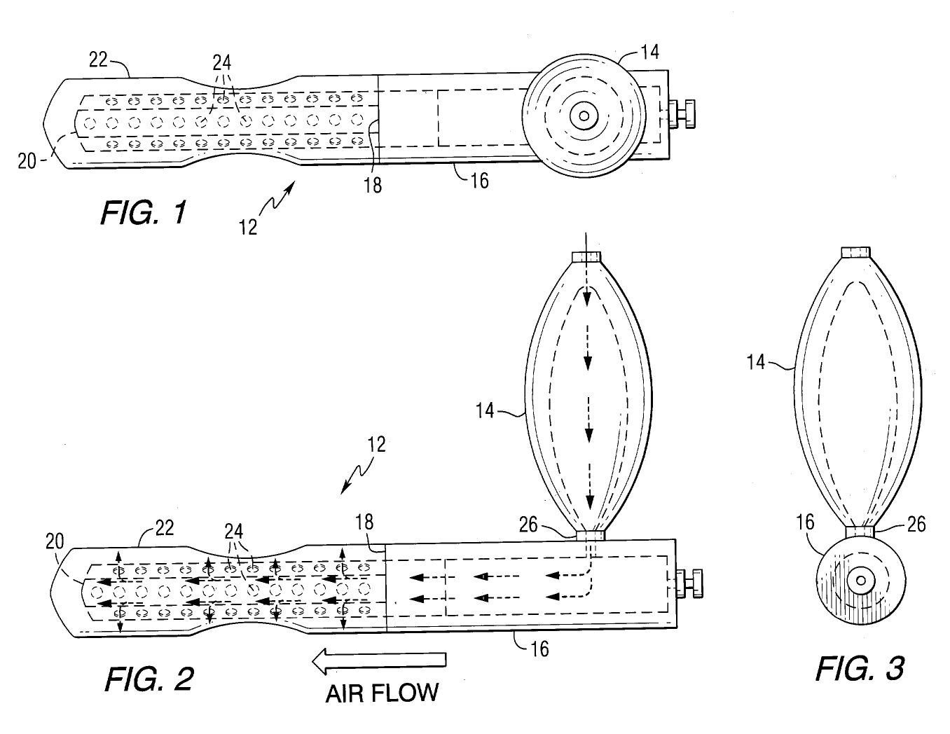

FIG. 1 is a plan view of a device according to the invention;

FIG. 2 is a sectional view of the device of FIG. 1;

FIG. 3 is an end view of the device of FIGS. 1 & 2;

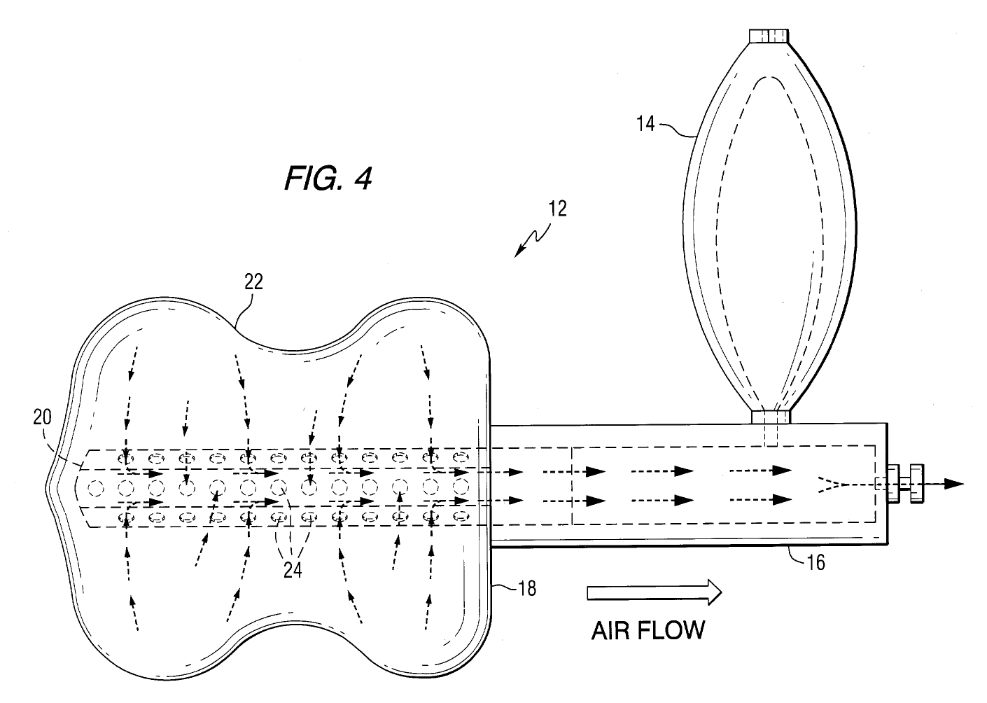

FIG. 4 shows a sectional view of the device of FIG. 1, with the extension member in an expanded manner but not to scale;

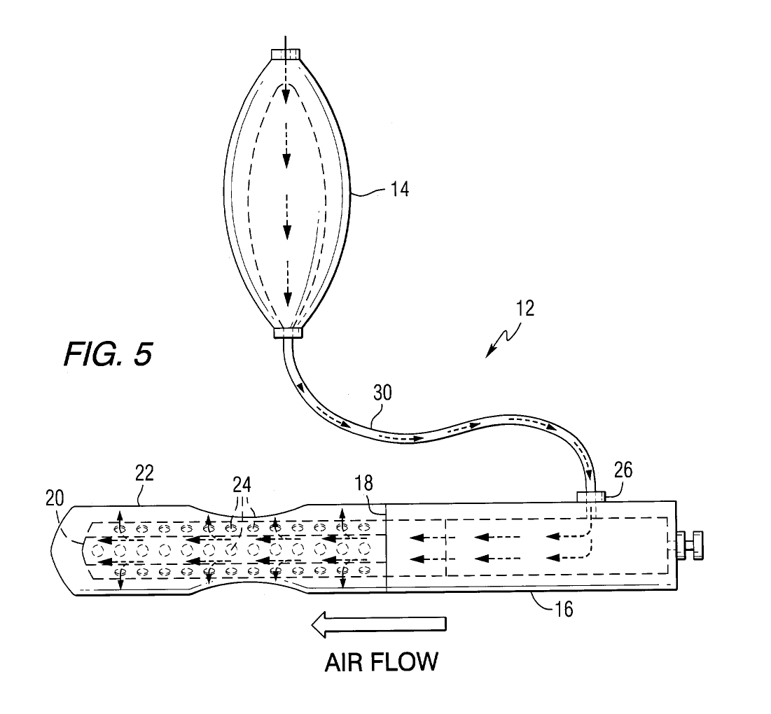

FIG. 5 depicts a sectional view with a tubular connection for the rigid chamber to a fluid pumping mechanism;

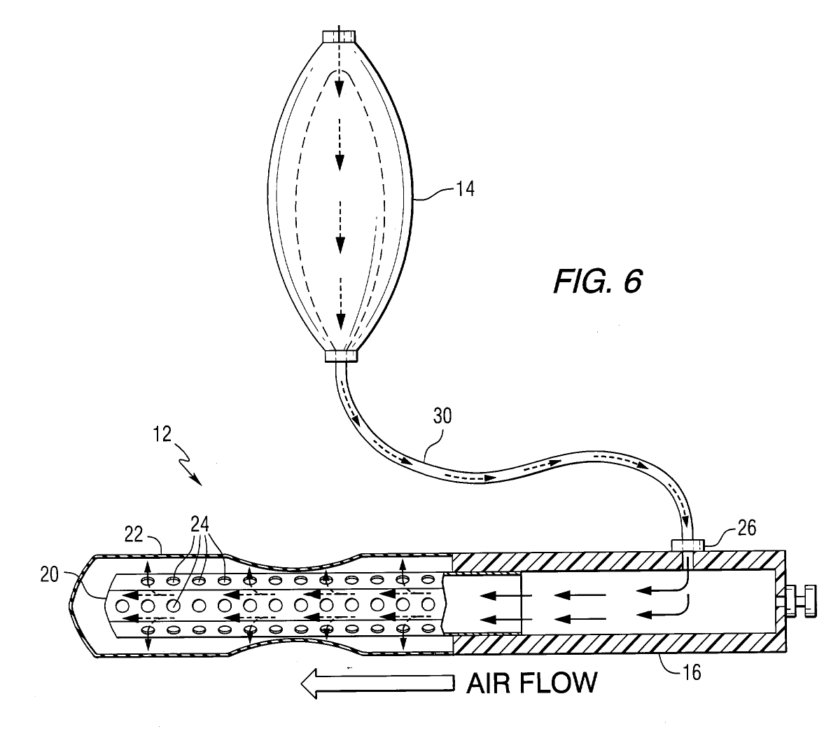

FIG. 6 is an alternative embodiment of FIG. 5 according to the invention.

In accordance with the invention and with reference to FIGS. 1 to 3, a device 12 is provided for regulating and controlling urinary incontinence. The device comprises a fluid pumping mechanism 14, for pumping fluids, preferably air. The pumping mechanism is interconnected directly to a rigid chamber 16. The rigid chamber is of cylindrical shape and is provided with an outlet 18.

An extension member 22 is connected by an adhesive to the outlet 18 of the rigid member 16. The extension member includes an outer wall 22 made of a flexible material, preferably of rubber in the form of a soft pliable cylindrical sleeve 22, and adapted and shaped for ease of insertion into a woman's vagina, so that when the soft pliable cylindrical sleeve 22 located within the woman's vagina enables the pelvic muscles to be exercised and strengthened and toned to regulate and control urinary incontinence.

Advantageously, a centering groove 23 (shown in FIGS. 4, 5, & 6) on the external side wall 22 is adapted to position the cylindrical extension member within the vagina with respect to the device 12. This provides a custom fit of the external side wall 22 to the floor muscles of a woman's vagina.

Suitably, the cylindrical extension member has a central tubular piece 20 with perforations 24 therein for proper distribution of air when the pumping mechanism 14 is used.

Preferably, a regulating and control valve is positioned within the air flow path between the pumping mechanism 14 and the central tubular piece 20. For convenience of operation the regulating valve 26 is located external to the rigid chamber 16.

With reference to FIG. 4, the cylindrical extension member 22 is depicted in an expanded form (not to scale) for exercise purpose of the pelvic muscles. The central portion 23 which forms the centering grove is formed with at least an extra layer of pliable rubber. This helps to increase resistance during inflation and forms the groove ideally suited for muscle placement.

In accordance with another aspect of the invention, FIGS. 5 and 6 depict a tubular connection in the form an extension tube 30 with the rigid member 16.

Alternatively, the rigid member 16 may be connected to the extension member 22 by a flanged connection which is not shown.

In accordance with another form of the invention a method of using the device. Since the extension member is of a flexible smooth rubber like material and cylindrically shaped, it could be easily inserted and inflated within the vagina and located and positioned for regular exercise and muscle toning, thus assist to regulate and control urinary incontinence.

This device is of considerable advantage over known exercising devices in that during resistance exercise, the muscles of the pelvic floor often become engorged with blood. The swelling in the muscles often makes it difficult to extract rigid exercise devices. This device allows the user to deflate the sleeve before extracting the device, therefore, swelling of the pelvic floor muscles is not a problem when using this device.

This device is completely sealed with no exposed or separate pieces and can be cleaned under the water tap.

Finally, this device is completely self-contained and works with air pressure. It eliminates severe inherent problems of other exercise devices. It has infinite variations in resistance and size. Because of the inflatable cover it will custom fit perfectly to any woman. It works the entire muscle circumference, and will not become lodged in place by muscle spasms and pinch during exercise or removal. Air is pumped into the flexible soft rubber cover which expands while retaining the muscle in the groove. The amount of pumping determines the resistance in performing the exercise. When finished, press the air release button and the rubber cover deflates for removal.

1. A device, for regulating and controlling urinary incontinence, comprising a fluid pumping means; a rigid chamber interconnected to the fluid pumping means having an outlet for fluid; an extension member connected to said outlet for fluid flow, characterized in that said extension member is of a flexible material and substantially cylindrical in shape and adapted for insertion into a woman's vagina, so that the cylindrical extension member when located within the woman's vagina enables pelvic muscles to be exercised and toned.

2. A device as claimed in claim 1 characterized in that a centering groove is provided and adapted to position the cylindrical extension member within the vagina with respect to the device, and the floor muscles of a woman's vagina.

3. A device as claimed in claims 1 & 2 characterized in that the cylindrical member has a central tubular piece with perforation therein for the distribution of fluid against the internal sidewall of the cylindrical extension member, so as to form a custom fit with respect to the pelvic floor muscles of the vagina.

4. A device as claimed in claim 3, characterized in that a regulating valve, is positioned within the fluid flow path between the pumping mechanism and the central tubular piece.

5. A device as claimed in claim 4, characterized in that the regulating valve is positioned external to the rigid chamber.

6. A device as claimed in claim 5, characterized in that the fluid pumping means is connected by a tubular extension member to the rigid chamber, and the regulating valve is positioned in the tubular extension member.

7. A device as claimed in claim 1, characterized in that the fluid may include air, water or liquid mixtures.

8. A device as claimed in claim 1, characterized in that the outlet extension member is formed of a flexible material may be constituted by a soft and pliable rubber or like material as an external sleeve.

9. A device as claimed in claim 8, characterized in that a central portion which forms the centering groove is formed with at least an extra layer of pliable rubber, so as to increase resistance during inflation and forms the centering groove for muscle placement.

10. A device as claimed in claim 1, characterized in that the rigid chamber is connected to the extension member by an adhesive.

11. A device as claimed in claim 1, characterized in that the rigid member is formed with a flange with a corresponding flange provided on the extension member adapted to form a connecting piece between the rigid chamber and the extension member.

12. A method, for controlling and regulating urinary incontinence characterized by the steps of insertion of an exercise device, comprising a fluid pumping means, a rigid chamber interconnected to the fluid pumping means have an outlet for fluid; an extension member connected to said outlet for fluid flow, characterized in that said extension member is of a flexible material and substantially cylindrical in shape and adapted for insertion into a woman's vagina, so that the cylindrical extension member when located within the woman's vagina enables pelvic muscles to be exercised and toned.

13. A method as claimed in claim 12, characterized by centering the device, adjusting the fluid pressure to form a custom fit of the extension member within a woman's vagina so as to provide resistance against pelvic floor muscles during exercise, and to adjust the fluid pressure.
